(19) United States
(12) Patent Application Publication
(10) Pub. No.: US 2009/0124570 A1
(43) Pub. Date: May 14, 2009

(54) Methods and Compositions for Facilitating Tissue Repair and Diagnosing, Preventing, and Treating Fibrosis

(75) Inventors: Sergei P. Atamas, Ellicott City, MD (US); Irina Luzina, Ellicott City, MD (US)

Correspondence Address: EDELL SHAPIRO & FINNAN , LLC, 1901 RESEARCH BLVD, SUITE 400, ROCKVILLE, MD, 20850, US

(73) Assignee: University of Maryland, Baltimore, Baltimore, MD (US)

(21) Appl. No.: 12/261,113

(22) Filed: Oct. 30, 2008

Related U.S. Application Data
(60) Provisional application No. 60/986,399, filed on Nov. 8, 2007.

(51) Int. Cl. A61K 31/7105

(52) U.S. Cl. 514/44

(57) ABSTRACT

Compositions and methods for facilitating tissue repair by enhancing the actions of BIGH3 or at least one of its downstream effector molecules in injured tissue, and for the diagnosis, prophylactic and therapeutic treatment of fibrosis by inhibiting the actions of BIGH3 or at least one of its downstream effector molecules, such as PU.1 transcription factor and MMP14. Other disclosed methods include methods of screening and/or identifying compounds useful for facilitating tissue repair, treating fibrosis, or for altering the accumulation or deposition of collagen, comprising contacting BIGH3 or its downstream effector molecules, such as PU.1 or MMP14, with a substance and subsequently determining the effects of the substance on the activity of BIGH3, PU.1, or MMP14.

### CROSS REFERENCE TO RELATED APPLICATIONS

The present invention claims priority to U.S. Provisional Patent Application No. 60/986,399, filed Nov. 8, 2007, which is incorporated herein by reference in its entirety.

### STATEMENT REGARDING FEDERALLY-SPONSORED RESEARCH

This work was sponsored at least in part by Grant No. 1RO1HL074067 from The National Institutes of Health, and a Veterans Administration Merit Review Type 1 award. The government has certain rights in the invention.

### BACKGROUND OF THE INVENTION

1. Field of the Invention

This invention relates to the facilitation of tissue repair by delivering BIGH3 to injured tissue, or otherwise enhancing the levels of BIGH3 in injured tissue, and to the diagnosis, prophylactic and therapeutic treatment of fibrosis by blocking BIGH3 activity in injured tissue, or otherwise decreasing the levels of BIGH3 in injured tissue, and more particularly in tissue prone to fibrosis or fibroproliferative disease. Methods of identifying compounds that modulate the activation or expression of BIGH3 and/or its effector molecules are also disclosed.

2. Description of the Background Art

Fibroblasts are the major cell type responsible for the synthesis of collagen, a fibrous protein essential for maintaining the integrity of the extracellular matrix found in the dermis of the skin and other connective tissues. The production of collagen is a finely regulated process, and its disturbance may lead to the development of tissue fibrosis. The formation of fibrous tissue is part of the normal healing process after injury, including injury due to surgery. However, in some circumstances there is an abnormal accumulation of fibrous material such that it interferes with the normal function of the affected tissue.

Scar tissue serves only a structural role, but does not contribute to the function of the organ in which it appears. For example, as fibrotic scar tissue replaces heart muscle damaged by hypertension, the heart becomes less elastic and thus less able to do its job. Similarly, pulmonary fibrosis causes the lungs to stiffen and impairs lung function. Fibrotic growth can proliferate and invade healthy surrounding tissue, even after the original injury heals. In most cases fibrosis is a reactive process, and several different factors can apparently modulate the pathways leading to tissue fibrosis. Such factors include the early inflammatory responses, local increase in fibroblast cell populations, modulation of the synthetic function of fibroblasts, and altered regulation of the biosynthesis and degradation of collagen.

Stimulation of fibroblast activity is involved in the development of fibrotic conditions, including spontaneous and induced conditions. Abnormal accumulation of collagen in the extracellular matrix, resulting from excessive fibroblast proliferation and/or collagen production, can cause fibrosis of a number of tissues including the skin. Many common debilitating diseases, such as liver cirrhosis and pulmonary fibrosis, involve the proliferation of fibrous tissue as do certain skin diseases such as scleroderma, and the formation of adhesions, keloids, and hypertrophic scars.

Macrophages are important functional contributors to normal wound healing/repair process; they participate in the clearance of apoptotic debris that accumulates as a result of primary injury and subsequent inflammatory response. Clearance of apoptotic debris is a major non-phlogistic function of macrophages; ingestion of apoptotic debris causes dramatic phenotypic changes in these cells. Of particular importance, the production of TGF-β by macrophages is accelerated following phagocytosis of apoptotic debris, suggesting potential anti-inflammatory and pro-fibrotic effects of this phenomenon. See M. L. Huynh et al. (2002) J Clin Invest. 109:41-50; Y. Q. Xiao et al. (2002) J Biol Chem. 277:14884-14893; P. P. McDonald et al. (1999) J Immunol. 163:6164-6172; V. A. Fadok et al. (1998) J Clin Invest. 101:890-898; S. P. Atamas & B. White (2003) Cytokine Growth Factor Rev. 14:537-550.

Based on these observations, it is reasonable to hypothesize that tissue fibrosis may be a consequence of disturbed clearance mechanisms of apoptotic debris by macrophages. Fibrosis, often viewed as an exaggerated repair process, is a major debilitating factor and a cause of death in patients with various diseases. S. P. Atamas & B. White (2003) Cytokine Growth Factor Rev. 14:537-550. Although macrophages may not be absolutely necessary for wound healing in macrophageless (PU.1 null) mice (apoptotic debris in the wounds is cleared by “stand-in” fibroblast phagocytes in such animals), the wounds heal with significantly less inflammation, lower levels of TGF-β, and less fibrosis in the absence of macrophages. P. Martin et al. (2003) Curr Biol. 13:1122-1128. Macrophages appear to be intimately involved in the regulation of tissue fibrosis in the lung, kidney, and liver; the apoptotic mechanisms are often involved in the mechanism of tissue fibrosis in the lung, kidney, and liver. See, e.g., H. Y. Reynolds (2005) Am J Respir Crit Care Med. 171:98-102; A. Prasse et al. (2006) Am J Respir Crit Care Med. 173:781-792; J. Zhang-Hoover et al. (2000) Immunology 101:501-511; J. Yamate et al. (2002) Vet Pathol. 39:322-333; F. Y. Chow et al. (2004) Nephrol Dial Transplant. 19:2987-2996; J. S. Duffield et al. (2005) J Clin Invest. 115:56-65; L. Wang et al. (2006) Am J Physiol Lung Cell Mol Physiol. 290:L695-L702; N. G. Docherty et al. (2006) Am J Physiol Renal Physiol. 290:F4-F13; A. Canbay et al. (2002) Gastroenterology 123:1323-1330.

Little is known about pro- and anti-fibrotic regulation by macrophages in relation to phagocytotic clearance of apoptotic debris. It is unclear whether TGF-β production following the uptake of apoptotic debris by macrophages is sufficient to drive tissue fibrosis, or whether more complex mechanisms, such as so-called alternative macrophage activation or yet unknown novel mechanisms are necessary. A. L. Mora et al. (2006) Am J Respir Cell Mol Biol. 35:466-473. Of note, the levels of TGF-β production by macrophages following phagocytosis of apoptotic debris are relatively low (within 100 pg/ml, see V. A. Fadok et al. (1998) J Clin Invest. 101:890-898), and thus are insufficient for direct activation of collagen production in fibroblasts. V. M. Kahari et al. (1990) J Clin Invest. 86:1489-1495; A. Fine & R. H. Goldstein (1987) J Biol Chem. 262:3897-3902.

Conventional treatment of most fibrosis-related disorders frequently involves corticosteroids, such as prednisone, and/or other medications that suppress the body's immune system. The goal of current treatment regimens is to decrease lung inflammation and subsequent scarring. Responses to currently available treatments are variable, and the toxicity and side effects associated with these treatments can be serious. Indeed, only a minority of patients respond to corticosteroids alone, and immune suppression medications are often used in combination with corticosteroids. Specific treatment of fibrosis has also been difficult, because although TGF-β is considered to be the central profibrotic cytokine, it is not a good target for the treatment of fibrosis because of its ubiquitous and systemic regulatory effects on the immune system and in connective tissue.

### SUMMARY OF THE INVENTION

The present invention relates to methods and compositions for promoting regeneration or repair of a tissue in a mammal in need of treatment thereof, said method comprising enhancing the activity of transforming growth factor beta induced (BIGH3) and/or its effector molecules.

The present invention also relates to methods and compositions for treating fibrosis in a subject in need of treatment thereof, the method comprising inhibiting the activity of BIGH3. In particular, it relates to a method of treating a disorder that results in fibrosis or sclerosis, in a subject in need of such treatment, comprising administering a composition comprising an inhibitor of BIGH3 activity, in an amount effective to reduce the rate of fibrosis or sclerosis. Further provided is a method of preventing fibrosis or sclerosis in a subject in need of such treatment, comprising administering a composition comprising an inhibitor of BIGH3 activity, in an amount effective to reduce the formation of fibrotic or sclerotic tissue that would occur in the absence of such treatment.

Also provided by the present invention are methods of identifying compounds useful for the treatment of fibrosis, comprising a) providing a test substance to a cell, where the cell possesses BIGH3 activity, b) measuring the amount of BIGH3 activity in the test cell; and c) comparing the amount of BIGH3 activity in a control cell, the control cell having not been provided the test substance, with the amount of BIGH3 activity, where a decrease in the amount of BIGH3 activity in the test cell, compared to the amount of BIGH3 activity in the control cell indicates that the test substance is useful for treating, preventing or preventing the progression of fibrosis.

Additional advantages and features of the present invention will be apparent from the following detailed description, drawings and examples, which illustrate preferred embodiments of the invention.

### BRIEF DESCRIPTION OF THE DRAWINGS

FIG. 1 depicts the regulation of collagen levels in macrophage-fibroblast co-cultures. Primary fibroblasts (Fib) were cultured with or without THP-1-derived macrophages (TDM). The macrophages were or were not exposed to apoptotic (Apopt) or necrotic (Necr) Jurkat cells. Panel A is a bar graph showing fold change in total (closed bars) or collagenase-sensitive (open bars) levels of metabolic incorporation of ¹⁴C-proline (mean CPM±SD of quadruplicate cultures). Significant increases in ¹⁴C-proline incorporation (p<0.05) are indicated with asterisks. Panel B shows representative Western blotting results for collagen type I in these co-cultures.

FIG. 2 is a bar graph illustrating the regulation of collagen levels in co-cultures of monocytes-derived macrophages (MDM) or alveolar macrophages (AM) and fibroblasts. The bars represent fold change in total (closed bars) or collagenase-sensitive (open bars) metabolic incorporation of ¹⁴C-proline (mean CPM±SD of quadruplicate cultures). The collagen production was upregulated when fibroblasts were co-cultured with MDM or AM alone and the levels of collagen increased further significantly when MDM have ingested apoptotic cells (Apopt). The levels of collagen did not change in co-cultures of fibroblasts with AM that have ingested apoptotic cells. Significant increases in ¹⁴C-proline incorporation (p<0.05) are indicated with asterisks.

FIG. 3 depicts the effect of macrophage-derived soluble factors on ¹⁴C-proline incorporation by primary fibroblasts. The bars represent fold change in total (closed bars) or collagenase-sensitive (open bars) metabolic incorporation of ¹⁴C-proline (mean CPM±SD of quadruplicate cultures). In Panel A, macrophages that have ingested apoptotic debris were separated from fibroblast monolayers with Transwell membranes. In Panel B, the medium conditioned by macrophages that have ingested apoptotic cells [(TDM+Apopt)sup] was transferred into fibroblast cultures. Thus, direct contact between macrophages and fibroblasts is not necessary to upregulate the collagen production; the effect is mediated, at least in part, by soluble factors produced by macrophages.

FIG. 4 is a bar graph illustrating the production of total TGF-β1 in cultures of monocyte-derived macrophages (MDM) after 2 h or 24 h of exposure to apoptotic cells (Apopt) measured by ELISA, mean pg/ml values ±SD of triplicate cultures. Levels of active TGF-β were below detection level in these assays.

FIG. 5 illustrates BIGH3 (TGFBI) protein and mRNA production by macrophage cultures. Panel A shows that the steady-state levels of BIGH3 mRNA increased significantly in THP-1 derived (TDM) and monocyte-derived macrophages (MDM) cultures following ingestion of apoptotic debris for 2 h, but not in alveolar macrophages (AM) cultures. Significant changes (p<0.05) are indicated with asterisks. Panel B shows fold changes in the steady-state levels of BIGH3 mRNA (measured by reverse transcriptase—real time PCR) in THP-1 derived macrophage cultures following exposure to apoptotic debris for indicated times. Significant changes (p<0.05) are indicated with asterisks. Panels C and D show Western blotting for BIGH3 protein in THP-1 derived (C) and alveolar (D) macrophage cultures.

FIG. 6 depicts time and dose-dependent effects of recombinant human BIGH3 (rhBIGH3) on collagen protein levels in primary lung fibroblast cultures. Collagen levels were measured by metabolic incorporation of ¹⁴C-proline (Panels A, C), or using Western blotting technique (Panels B, D). Significant increases in ¹⁴C-proline incorporation (p<0.05) are indicated with asterisks. In Panels A and B, fibroblasts were activated for 48 h with increasing concentrations of rhBIGH3 as indicated. In Panel C, fibroblast cultures were incubated for indicated times without or with 100 ng/ml rhBIGH3. In Panel D, fibroblasts were cultured for 72 h, with 100 ng/ml rhBIGH3 added to the cultures for the final 24, 48, or 72 h as indicated.

FIG. 7 illustrates MMP14 mRNA (A) and protein (B) levels in primary fibroblasts. In Panel A, mRNA was purified from control or BIGH3-stimulated fibroblasts and reverse transcribed into cDNA; real-time PCR amplification was performed using primers for a housekeeping gene GAPDH and for MMP14 gene. The amplification curves for GAPDH corresponding to control and BIGH3-stimulated fibroblasts closely overlapped, whereas amplification of MMP14 template in cDNA from BIGH3-stimulated fibroblasts occurred two cycles later, suggesting approximately 2²=4 fold lower levels of MMP14 mRNA. In Panel B, primary fibroblasts were cultured for indicated times without or with 300 ng/ml of rhBIGH3. Lysates were normalized for total protein, and Western blotting assays were conducted using anti-MMP14 antibody.

FIG. 8 depicts the effect of MMP14 or p53 overexpression, or PU.1 siRNA inhibition on the expression levels of MMP14 and collagen. Fibroblast cultures were transfected with MMP14-encoding or p53-encoding plasmid, or with PU.1 siRNA as indicated. Western blotting assays were performed using antibodies against MMP14 or collagen.

FIG. 9 illustrates a proposed outline of the sequential regulation of collagen expression in fibroblasts by macrophages following exposure to apoptotic cells.

### DETAILED DESCRIPTION

Reference will now be made in detail to the presently preferred embodiments of the invention, which, together with the drawings and the following examples, serve to explain the principles of the invention. These embodiments are described in sufficient detail to enable those skilled in the art to practice the invention, and it is to be understood that other embodiments may be utilized, and that structural, biological, and chemical changes may be made without departing from the spirit and scope of the present invention. Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, the preferred methods, devices, and materials are now described.

One skilled in the art may refer to general reference texts for detailed descriptions of known techniques discussed herein or equivalent techniques. These texts include Current Protocols in Molecular Biology (Ausubel et al., eds., John Wiley & Sons, N.Y., and supplements through July 2007), Current Protocols in Immunology (Coligan et al., eds., John Wiley & Sons, N.Y., and supplements through August 2007), Current Protocols in Pharmacology (Enna et al., eds., John Wiley & Sons, N.Y., and supplements through June 2007), The Pharmacological Basis of Therapeutics (Goodman & Gilman, 11^{th} ed., 2006), and Remington: The Science and Practice of Pharmacy (Lippincott Williams & Wilkins, 21st edition (2005)) for example.

Phagocytic clearance of apoptotic cells by macrophages is an essential part in the resolution of inflammation, and coincides with activation of repair mechanisms, including accumulation of extracellular matrix. We conducted a series of experiments to determine if a link exists between clearance of apoptotic debris and accumulation of extracellular matrix. We investigated the effects of phagocytosis of apoptotic or necrotic cells by macrophages on the rate of collagen production by primary fibroblasts in cell culture, and the molecular mechanisms of such effects.

We measured the production of collagen in primary fibroblasts co-cultured with macrophages, and determined that phagocytosis of apoptotic but not necrotic debris by monocyte-derived (not alveolar) macrophages stimulates collagen production in co-cultures with primary fibroblasts. This regulation is mediated by transforming growth factor beta induced (TGFBI) protein, which is also called keratoepithelin, Beta-IG-H3 (BIGH3), RGD-containing collagen-associated protein (RGD-CAP), CDB1, CDG2, LCD1, CDGG1, CSD, etc. BIGH3 is a 68 kDa extracellular matrix protein encoded by a gene located on chromosome 5 (5q31.1), and is an adhesive molecule that interacts with integrins. Its expression is highly induced by TGF-β and SPARC (secreted protein, acidic and rich in cysteine). BIGH3 mediates keratinocyte differentiation, adhesion and migration, and mutations in BIGH3 are associated with corneal dystrophy, corneal amyloid deposition, and intracraneal aneurisms.

Experimental results indicated that direct contact between macrophages and fibroblasts was not required for collagen upregulation. Macrophages produced TGF-β following ingestion of apoptotic cells, but the levels of this cytokine were lower than those required for a significant upregulation of collagen. Simultaneously, the levels of BIGH3 mRNA and protein were increased. In contrast, primary alveolar macrophages stimulated collagen production without exposure to apoptotic cells; there was no further increase in the levels of BIGH3 mRNA or protein, or collagen, after ingestion of apoptotic cells.

We also determined that the production of BIGH3 by macrophages leads to upregulation of collagen protein but not mRNA in primary fibroblasts. Stimulation of primary fibroblast cultures with recombinant BIGH3 led to downregulation of matrix metalloproteinase 2 (MMP2) and matrix metalloproteinase 14 (MMP14) levels, decreased DNA binding by p53, increased DNA binding by PU.1, and upregulation of collagen protein but not mRNA levels. Consistent with these observations, overexpression of MMP14 or p53, as well as siRNA-mediated inhibition of PU.1, caused elevated expression of MMP14 and a decline in collagen in primary fibroblast cultures. In conclusion, monocyte-derived but not alveolar macrophages produce BIGH3 following ingestion of apoptotic cells, leading to downregulation of MMP14 levels in fibroblasts through a mechanism involving p53 and PU.1, and to subsequent accumulation of collagen. This effect of BIGH3 is mediated by a decrease in the levels of MMP14 mRNA and protein in a p53-dependent and PU.1-dependent fashion.

Thus, we have identified a novel mechanism by which macrophages that ingest apoptotic cells can regulate normal wound healing and, if exaggerated, possibly fibrosis. This mechanism is the basis of the present invention, which concerns methods and compositions for facilitating tissue repair and wound healing, for diagnosing fibrosis and related conditions, and for the prophylactic and therapeutic treatment of fibrosis and related conditions by altering the activity of BIGH3 or at least one of its downstream effector molecules, such as, but not limited to, MMP2, MMP14, PU.1 transcription factor, or p53 transcription factor, in the tissue of interest.

### A. BIGH3 and its Role in Tissue Repair

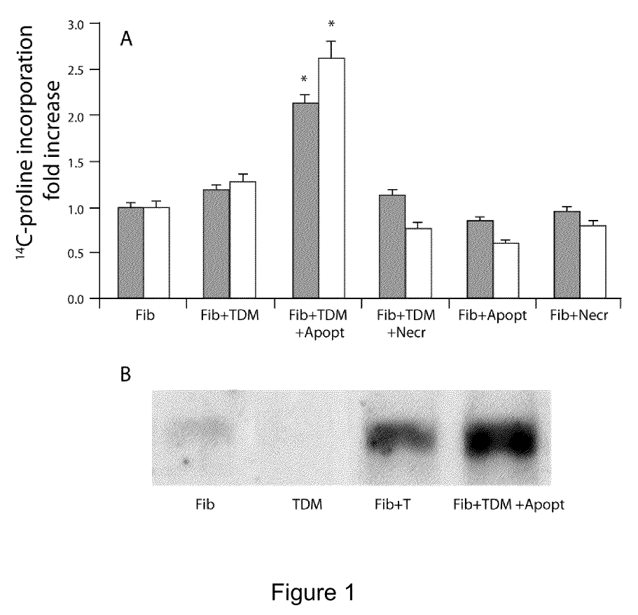

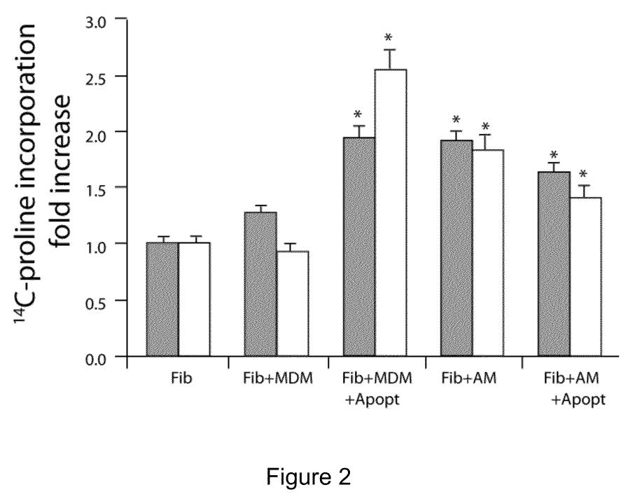

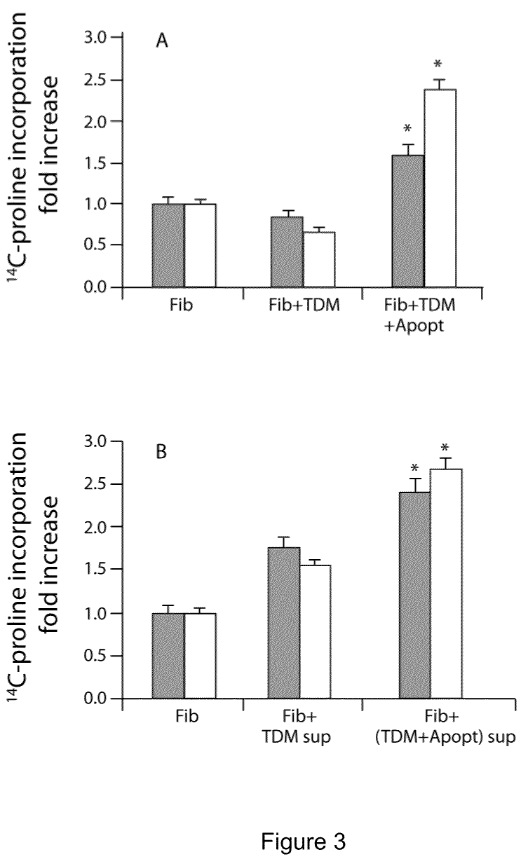

As further described in the following Examples, our experiments investigated macrophage involvement in the regulation of extracellular matrix following ingestion of apoptotic cells. Macrophages derived from a monocytic cell line THP-1 (see FIG. 1), as well as macrophages derived from primary monocytes (see FIG. 2), stimulated upregulation of collagen protein levels in co-cultures with fibroblasts, following ingestion of apoptotic cells. In contrast, primary alveolar macrophages stimulated upregulation of collagen levels even without exposure to apoptotic cells, and this effect was not further increased following ingestion of apoptotic debris (see FIG. 2). The effect of ingestion of apoptotic cells by macrophages on collagen production by fibroblasts in co-cultures was preserved if the macrophages and fibroblasts were separated by a Transwell membrane (see FIG. 3A), suggesting that soluble factors produced by macrophages mediated the effect on collagen levels. If macrophages were exposed to apoptotic cells separately, and the conditioned medium was then transferred into the cultures of the primary fibroblast monolayers, the effect was still preserved (see FIG. 3B), further supporting the notion that soluble factor(s) were produced by macrophages following ingestion of apoptotic cells, and that those soluble factors regulated accumulation of collagen.

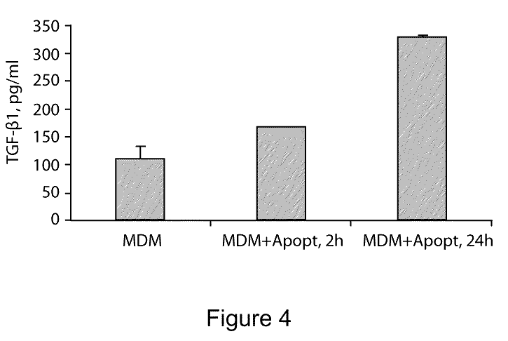

An obvious candidate for such a soluble factor would be TGF-β, a factor whose expression in macrophages does increase following ingestion of apoptotic debris, and that is a known potent profibrotic factor. See V. A. Fadok et al. (1998) J Clin Invest. 101:890-898; S. P. Atamas & B. White (2003) Cytokine Growth Factor Rev. 14:537-550. Indeed, ELISA assays revealed that total TGF-β was elevated in the macrophage culture supernatants following ingestion of apoptotic debris (see FIG. 4A). However, such an increase in TGF-β does not explain the observed increase in collagen for the following reasons. First, a concentration of at least 500 pg/ml of active TGF-β is required for upregulation of collagen production (as reported by I. G. Luzina et al. (2006) Am J Respir Cell Mol Biol. 35:298-305), whereas in these assays, total TGF-β did not exceed 325 pg/ml, and active TGF-β was not detectable. Second, TGF-β is known to upregulate collagen production transcriptionally, leading to elevated steady-state levels of collagen mRNA, but no such increase in COL1A2 mRNA was observed by real-time PCR in fibroblast-macrophage co-cultures following ingestion of apoptotic cells. Together, these observations suggest that an additional soluble factor may promote upregulation of collagen protein levels.

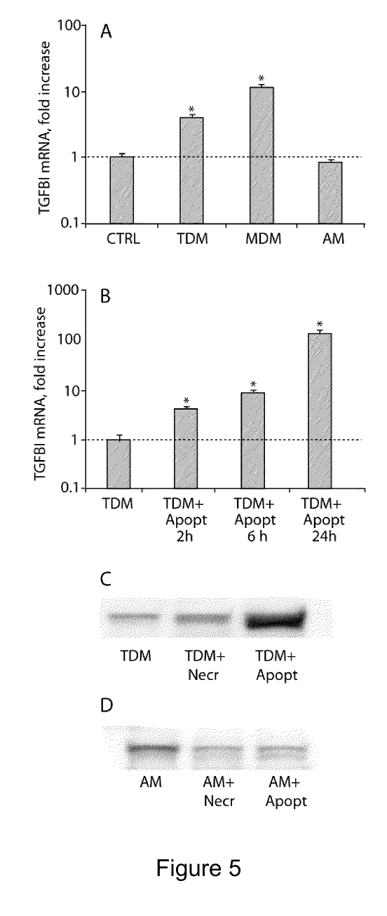

We then investigated this possibility by profiling of gene expression for cytokines and related factors performed in fibroblasts from co-cultures with macrophages. In three independent experiments, expression of mRNA for BIGH3 was increased. Real-time PCR experiments confirmed this observation in THP-1-derived and primary monocyte-derived macrophages, but not in alveolar macrophages (see FIG. 5A), in a time-dependent fashion (see FIG. 5B). Western blotting analyses for BIGH3 confirmed that its expression increased following ingestion of apoptotic but not necrotic cells (see FIG. 5C). This effect on BIGH3 expression was observed in THP-1-derived macrophages (see FIG. 5C) but not in alveolar macrophages (see FIG. 5D). Thus, expression of BIGH3 in macrophages and the effect on collagen accumulation in co-cultures with fibroblasts are both upregulated following ingestion of apoptotic cells in THP-1-derived and primary monocyte-derived, but not in alveolar macrophages (compare FIG. 5 with FIGS. 1, 2). These observations suggest that BIGH3 may be an important factor mediating the profibrotic effect of macrophages that ingested apoptotic cells. While producing BIGH3, and acting profibrotically similarly to alternatively activated macrophages, the macrophages that ingested apoptotic cells did not express other markers of alternative activation such as CD163 or CD206. See, e.g., A. Gratchev et al. (2001) Scand J Immunol. 53:386-392; E. Song et al. (2000) Cell Immunol. 204:19-28; V. Porcheray et al. (2005) Clin Exp Immunol. 142:481-489.

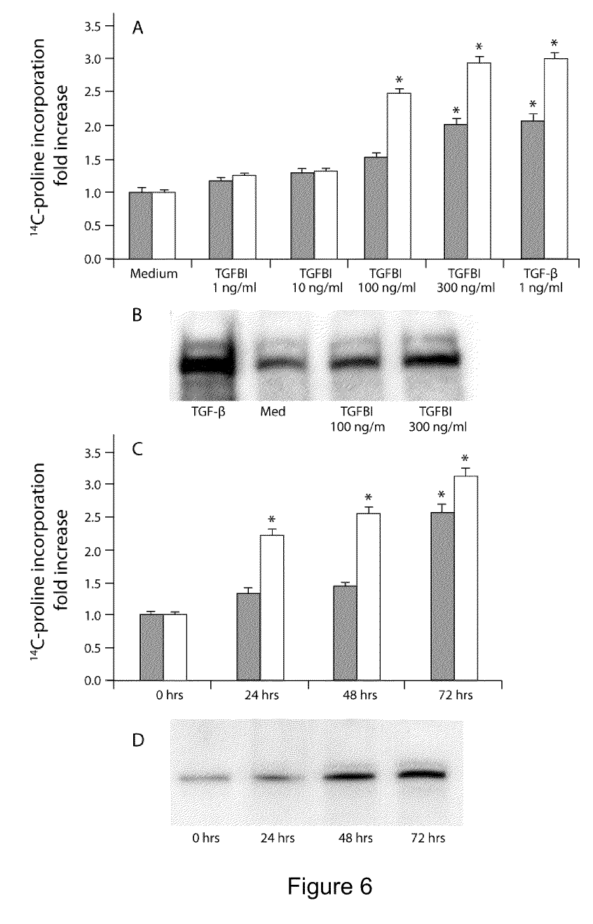

To investigate the possible profibrotic involvement of BIGH3, recombinant human (rh) BIGH3 was added to cultured primary fibroblasts and collagen levels assessed (see FIG. 6). Increases in collagen protein levels were observed in response to stimulation with rhBIGH3 in a dose- and time-dependent fashion (see FIG. 6). However, BIGH3 did not stimulate an increase in collagen mRNA according to real-time PCR data. Also, a collagen promoter-CAT reporter construct that has been previously described to respond to TGF-β activation, see I. G. Luzina et al. (2006) Am J Respir Cell Mol Biol. 35:298-305, was transfected into fibroblast and was responsive to TGF-β stimulation but non-responsive to BIGH3 stimulation. Finally, profiling of DNA binding by transcription factor arrays showed no increase in the activity of Smad3/4, Sp1, AP1, and Ets (factors known to regulate activity of the collagen gene promoter). Together, these observations suggest that BIGH3 regulates collagen levels through a mechanism that is different from transcriptional upregulation of the collagen gene expression.

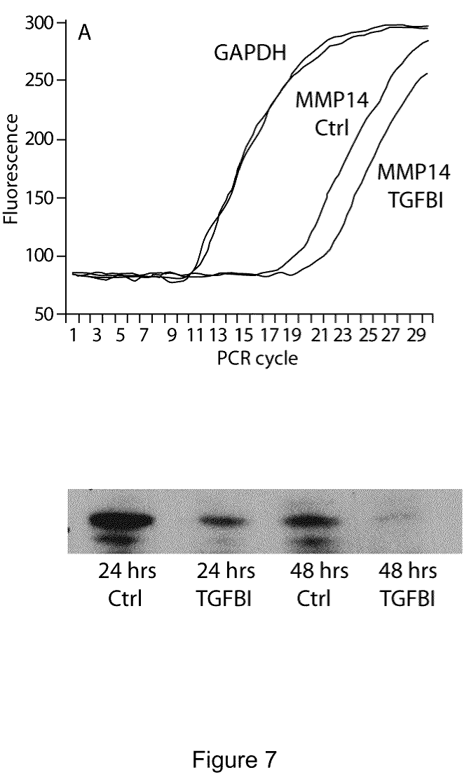

A possibility was considered that BIGH3 may regulate collagen turnover, e.g. by inhibiting the levels of matrix metalloproteinases in fibroblasts. To address such a possibility, real-time PCR experiments were conducted in fibroblasts that were or were not activated with rhBIGH3, to compare steady-state levels of mRNA for genes related to extracellular matrix. A real-time PCR-based system was utilized to simultaneously compare expression of 84 extracellular matrix relevant genes. The differences between BIGH3-activated and control cells were observed for MMP14 (also termed MT1-MMP), whose mRNA levels decreased four fold following activation with BIGH3 (see FIG. 7A). Western blotting assays revealed a similar decrease in MMP14 protein following stimulation of fibroblasts with BIGH3 (see FIG. 7B).

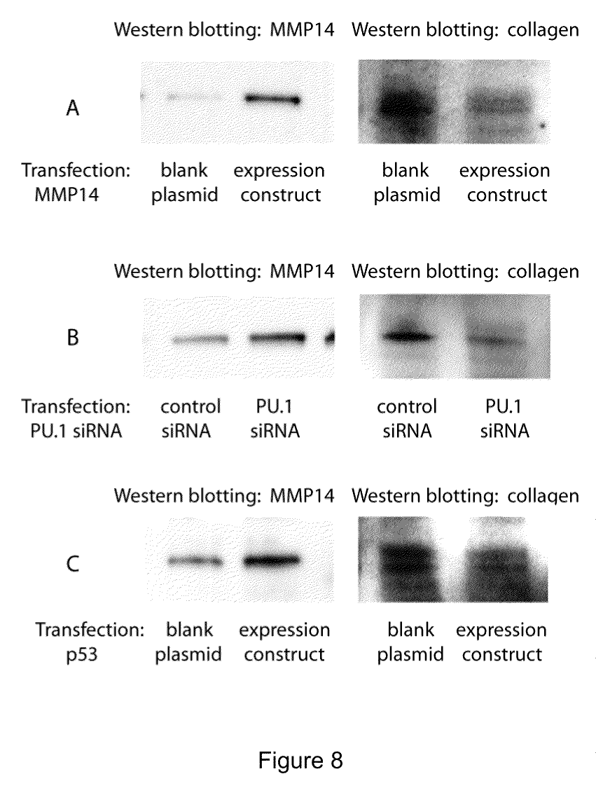

MMP14 is able to directly degrade various extracellular matrix components, including type I, II, and III collagens, gelatin, fibronectin, vitronectin, tenascin, entactin, and laminin-1, as well as to directly activate pro-MMP-2. H. Sato et al. (1994) Nature 370:61-65; A. Okada et al. (1997) J Cell Biol. 137:67-77. The loss of MMP14 leads to significant disturbances of connective tissue metabolism. Fibroblasts from MMP14-deficient mice completely lose the ability to degrade collagen fibrils, resulting in severe and progressive fibrosis in many tissues of MMP14-deficient mice. K. Holmbeck et al. (1999) Cell 99:81-92. In our study, overexpression of MMP14 in cultured fibroblasts caused downregulation of collagen levels (see FIG. 8A), confirming previous observations of MMP14 involvement in collagen turnover by fibroblasts. H. Lee et al. (2006) Mol Biol Cell. 17:4812-4826.

Because the levels of MMP14 decreased at both mRNA and protein levels, the possibility of transcriptional regulation of MMP14 by BIGH3 was considered. To assess possible transcription factors that might be involved in downregulation of MMP14, profiling of DNA binding activity by various transcription factors was assessed in BIGH3 stimulated fibroblasts in comparison with control fibroblasts. Experiments with transcription factor arrays revealed that out of 345 transcription factors analyzed, two factors significantly changed their DNA binding activity in fibroblasts in response to activation with BIGH3. Activity of PU.1 was increased and activity of p53 was decreased following fibroblast activation with BIGH3. Inhibition of PU.1 expression with siRNA in cultured fibroblasts led to an increase in MMP14 expression levels and a decrease in collagen levels (see FIG. 8B). This observation is consistent with the notion that PU.1 is a repressor of MMP14 expression, as stimulation with BIGH3 activates DNA binding by PU.1 and simultaneously causes a decrease in MMP14 expression (see FIG. 7). Overexpression of p53, a possible activator of MMP14 expression, led to a similar increase in MMP14 and decrease in collagen expression (see FIG. 8C). The latter observation is consistent with a previous report suggesting an inhibitory effect of p53 on TGF-β-stimulated collagen expression. See A. K. Ghosh et al. (2004) J Biol Chem. 279:47455-47463.

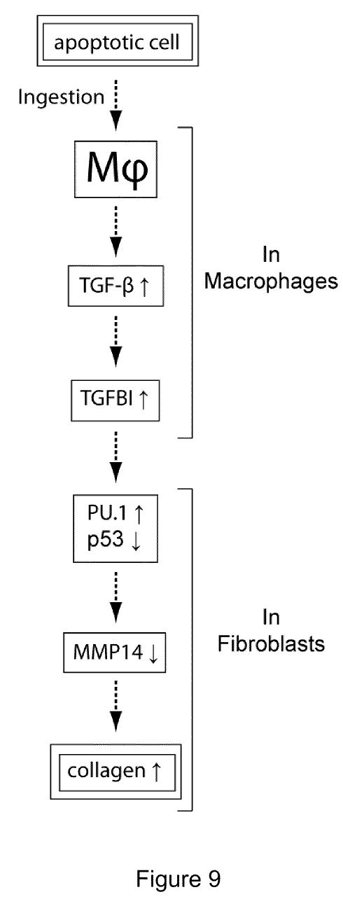

Based on the observations summarized above, a sequence of events connecting ingestion of apoptotic cells by macrophages with collagen accumulation in fibroblasts appears to be as presented in FIG. 9. Physiologically, the studied mechanisms are likely to be relevant to inflammation resolution and healing, and if exaggerated contribute to tissue fibrosis. Pharmacological targeting of this pathway is useful in therapies for delayed wound healing or for tissue fibrosis.

Thus, BIGH3 directly stimulates collagen production in fibroblasts by at least two distinct downstream effector molecules: MMP2 and MMP14. As used here, the phrase “effector molecule” is used to mean a molecule that is capable of generating a signal or subsequent message (second messenger) or capable of exerting a detectable intracellular or intercellular effect on the metabolism, gene expression or proliferation of a cell or group of cells, such as but not limited to tissue or an organ. An effector molecule is responsive to an “activator molecule”, i.e., the detectable effects of effector molecules are correlative, either directly or inversely, with the presence or absence of the “activator molecules.” As used herein, an “activator molecule” is a molecule or compound that directly or indirectly initiates or inhibits the activity of at least one effector molecule. For example, an activator molecule may initiate a cascade of intracellular or intercellular events and/or signals that ultimately leads to the activation or inactivation of an effector molecule, which, in turn, will affect the metabolism, gene expression or proliferation of a cell or group of cells. Accordingly, it is possible that a specific molecule may be considered to be an activator molecule as well as an effector molecule, relative to its position in a signaling cascade event. For example, the BIGH3 receptor and MMP14 would be effector molecules, relative to BIGH3; however, MMP14 may also be considered an activator molecule relative to a transcription factor, such as, but not limited to PU.1 and p53. In turn PU.1 would also be an effector molecule relative to BIGH3.

Accordingly, certain embodiments of the present invention relate to various methods of facilitating tissue repair of a wound or other injured tissue in a patient comprising delivering BIGH3 to injured tissue, or otherwise enhancing the levels of BIGH3 in injured tissue. In particular, certain embodiments relate to delivering or enhancing the activity of BIGH3 and/or its downstream effector molecules, including MMP2, MMP14, PU.1 and p53. These methods may include delivery of a pharmaceutical composition, gene delivery, administration of agonists, administration of antibodies, etc.

These methods can be used to promote tissue regeneration and/or wound healing in a variety of tissues. The methods are effective for enhancing tissue regeneration and wound healing in epithelial tissues, and for promoting regeneration of bone and/or cartilage tissues. For example, in certain embodiments, a pharmaceutical composition can be applied to an area where epithelium, bone or cartilage has been broken, torn or eroded due to injury or disease, to stimulate the regeneration and repair of the epithelium, bone or cartilage. The methods are particularly effective for treating tissues affected by periodontal disease, for example by applying a composition of BIGH3 to the affected gum tissue and periodontal ligament, thereby promoting regeneration of the gum tissue, of tooth tissues such as dentin and pulp, and of the connective tissue holding the tooth in place in the gum. The methods are also particularly effective for treating external wounds, including skin ulcers, burns and lesions and for regenerating connective tissue and/or bone.

Wound healing and tissue regeneration can be promoted by directly, locally applying an effective amount of a composition comprising BIGH3 and/or a downstream effector molecule to the affected tissue. The tissue can be external epithelial tissue, internal epithelial tissue, muscle, bone, cartilage, tendons, ligaments, or dental tissue, including gum tissue, dentin, pulp, cementum or periodontal ligature. Tissue can be soft tissue, such as muscles, fibrous tissues, fat, blood vessels, and synovial tissues, or hard tissue, such as bone, cartilage, periodontium, or ligaments. In a preferred embodiment, the tissue to be repaired is epithelium, bone or cartilage, or dental tissue. The wound or tissue damage to be repaired may be any wound, damage, or trauma such as, but not limited to, a wound, surgical incision, bony defect, bony fracture, or prosthetic implant. Depending on the type and location of the wound or tissue damage, administration of the composition can be by any suitable means, including, but not limited to topical, intravenous, intra-articular, parenteral, or the like. When surgery is indicated, the compositions can be administered prophylactically, e.g., prior to a scheduled surgery, or contemporaneous with (e.g., during, or shortly before or after) surgery, in order to facilitate tissue repair.

Certain other embodiments of the present invention relate to various methods of diagnosis, prophylactic treatment, and therapeutic treatment of fibrosis by blocking BIGH3 activity in injured tissue, or otherwise decreasing the levels of BIGH3 and/or its downstream effector molecules, including MMP2, MMP14, PU.1 and p53, in injured tissue, and more particularly in tissue prone to fibrosis or keloids. In a different embodiment, the invention provides methods of treating, preventing or preventing the progression of fibrosis in a patient comprising inhibiting the activity of BIGH3 in combination with inhibiting the activity of TGF-β. In one particular embodiment, the methods of inhibiting both BIGH3 and TGF-β may comprise administering a single active compound that inhibits both BIGH3 and TGF-β. In another particular embodiment, the methods of inhibiting both TGF-β and BIGH3 comprise coadministering more than one active compound, which may or may not be in admixture together. As used herein, the term “coadminister” is used to mean that each of at least two compounds are administered during a time frame wherein the respective periods of biological activity or effects overlap. Thus the term includes sequential as well as coextensive administration.

As used herein, the term “enhance” is used to mean that the treatment confers detectable increase in activity or effects of a molecule, and the term “inhibit” is used to mean that the treatment confers detectable decrease in activity or effects of a molecule, as compared to that of the untreated molecule. The detectable alteration may be complete, e.g., inhibition resulting in no detectable activity observed after treatment, or it may be partial, e.g., a partial increase in activity, or a partial decrease in activity. For example, if the activity of a particular molecule includes phosphorylation of target proteins or molecules, inhibiting would comprise detectably decreasing the phosphorylation of target molecules, and enhancing would comprise detectably increasing the phosphorylation of target molecules, which could directly or indirectly be assayed. Similarly, if the activity of a particular molecule includes DNA binding, inhibiting this activity would comprise modulating the DNA-binding molecule such that there is a detectable decrease in the binding of the molecule to nucleic acids, and enhancing this activity would comprise modulating the DNA-binding molecule such that there is a detectable increase in the binding of the molecule to nucleic acids, which could be directly or indirectly assayed.

Fibrosis is the formation of fibrous tissue, usually as a reparative or a reactive process. As used herein, “fibrosis” or “fibroproliferative disease” does not refer to the formation of fibrous tissue that is a normal part of an organ or tissue, but includes those disorders or disease states that are caused by or accompanied by the abnormal deposition of scar tissue, or by excessive accumulation of collagenous connective tissue. Thus a molecule that “promotes fibrosis” is a molecule that directly or indirectly contributes to the accumulation of collagenous tissue.

Examples of pathologic and excessive fibrotic accumulations include, but are not limited to, pulmonary fibrosis, asthma, adult respiratory distress syndrome (ARDS), chronic obstructive pulmonary disease (COPD), idiopathic pulmonary fibrosis (IPF), acute lung injury (ALI), pulmonary fibrosis due to infectious or toxic agents, such as radiation therapy or chemotherapy, pulmonary fibrosis due to particle inhalation, post-transplant pulmonary fibrosis, perirenal fascitis, glomerulonephritis (GN), diabetic nephropathy, renal interstitial fibrosis, renal fibrosis resulting from complications of drug exposure, HIV-associated nephropathy, transplant necropathy, retroperitoneal fibrosis, perivascular fibrosis in Systemic Lupus Erythematosus (SLE), obstruction-induced fibrosis in kidneys or spleen, benign prostate hypertrophy, fibrocystic breast disease, uterine fibroids, ovarian cysts, endometriosis, coronary infarcts, myocardial fibrosis, cerebral infarcts, congestive heart failure, dilated cardiomyopathy, myocarditis, vascular stenosis, progressive systemic sclerosis, polymyositis, scleroderma, dermatomyositis, Raynaud's syndrome, rheumatoid arthritis, musculoskeletal fibrosis, post-surgical adhesions, liver fibrosis, autoimmune hepatitis, cirrhosis including primary biliary cirrhosis, viral hepatitis including HIV- or Hepatitis C-induced hepatitis, real fibrotic disease, fibrotic vascular disease, e.g., atherosclerosis, varix, or varicose veins, scleroderma, Alzheimer's disease, diabetic retinopathy, glaucoma, proliferative vitreoretinopathy, fibrosis associated with ocular surgery, chronic transplant rejection, graft vs. host disease, radiation-induced fibrosis, and excessive or hypertrophic scar and/or keloid formation in the dermis occurring during wound healing resulting from trauma or surgical wounds, to name a few.

In a preferred embodiment, the disorder to be treated is a disorder that results in fibrosis or sclerosis, including but not limited to groups of disorders selected from the following: (1) skeletal muscle fibrosis, irradiation-induced fibrosis, autoimmune-related fibrosis, cardiovascular fibrosis, arteriosclerotic disorders, pulmonary fibrosis, adult respiratory distress syndrome, inflammatory disorders, scleroderma, cirrhosis, keloids, adhesions and hypertrophic scars; (2) skeletal muscle fibrosis associated with a condition selected from muscular dystrophy, denervation atrophy induced by neuromuscular disease, and traumatic injury-induced denervation atrophy; (3) cardiovascular fibrosis selected from left ventricular hypertrophy secondary to hypertension, fibrosis associated with myocardial infarction, fibrosis associated with ischemiareperfusion injury, and fibrosis associated with myocarditis; (4) dermal fibrosis; (5) keloid formation, hypertrophic scar formation, and adhesion formation; or (6) pulmonary fibrosis, pulmonary fibrosis due to adult respiratory distress syndrome and irradiation induced fibrosis.

Providing a therapy or “treating” refers to any indicia of success in the treatment or amelioration of an injury, pathology or condition, including any objective or subjective parameter such as abatement, remission, diminishing of symptoms or making the injury, pathology or condition more tolerable to the patient, slowing in the rate of degeneration or decline, making the final point of degeneration less debilitating, or improving a patient's physical or mental well-being. Those in need of treatment include those already with the disorder as well as those prone to have the disorder or those in whom the disorder is to be prevented. In the treatment of a fibroproliferative disease, a therapeutic agent may directly decrease the pathology of the disease, or render the disease more susceptible to treatment by other therapeutic agents. Preferred subjects for treatment include animals, most preferably mammalian species such as humans, and domestic animals such as dogs, cats and the like, subject to disease and other pathological conditions. A “patient” refers to a subject, preferably mammalian (including human).

### B. Pharmaceutical Compositions & Methods

Certain embodiments of the present invention relate to pharmaceutical compositions comprising one or more therapeutic agents, and methods of administering a therapeutically effective amount of one or more therapeutic agents, which are capable of prophylactic and/or therapeutic treatment of fibrosis and related conditions. The term “therapeutic agent” refers to any pharmaceutically acceptable acid, salt, ester, derivative, stereoisomer, pro-drug, or mixture of stereoisomers of a therapeutic agent, or to the therapeutic agent itself. Pharmaceutically acceptable acids, salts, esters, derivatives, stereoisomers, pro-drugs, and mixtures of therapeutic agents may also be used in the methods and compositions of the present invention. The therapeutic agents used herein can include BIGH3, MMP2, MMP14, PU.1 and p53, their agonists and antagonists, agents that increase or decrease their expression, antibodies and functional fragments thereof, etc.

The pharmaceutical compositions can be formulated according to known methods for preparing pharmaceutically useful compositions, and may include a pharmaceutically acceptable carrier. The carrier may be liquid, solid, or semi-solid, for example. Formulations are described in a number of sources which are well known and readily available to those skilled in the art. The physical and chemical characteristics of the compositions of the invention may be modified or optimized according to the skill in the art, depending on the mode of administration and the particular disease or disorder to be treated. The compositions may be in any suitable form, depending on the desired method of administration, and may be provided in unit dosage form, a sealed container, or as part of a kit, which may include instructions for use and/or a plurality of unit dosage forms.

The term “pharmaceutically acceptable” used herein refers to those modifications of the parent compound (acids, salts, esters, etc.) that do not significantly or adversely affect the pharmaceutical properties (e.g., toxicity, efficacy, etc.) of the parent compound. For example, exemplary pharmaceutically acceptable salts administrable by means of the compositions of this invention include chloride, iodide, bromide, hydrochloride, acetate, nitrate, stearate, palmoate, phosphate, and sulfate salts. Exemplary techniques for producing pharmaceutically acceptable derivatives include methylation, halogenation, acetylation, esterification, and hydroxylation.

As used herein, the term “therapeutically effective amount” means the total amount of each active component of the pharmaceutical composition or method that is sufficient to show a meaningful patient benefit, i.e., healing or amelioration of chronic conditions, a reduction in symptoms, an increase in rate of healing of such conditions, or a detectable change in the levels of MMP or other related proteinases in the treated or surrounding tissue. When applied to an individual active ingredient, administered alone, the term refers to that ingredient alone. When applied to a combination, the term refers to combined amounts of the active ingredients that result in the therapeutic effect, whether administered in combination, serially, or simultaneously.

Dosages of the pharmaceutical compositions can vary between wide limits, depending upon the disease or disorder to be treated, the age and condition of the individual to be treated, etc. Therapeutic efficacy and toxicity of the compositions may be determined by standard pharmaceutical, pharmacological, and toxicological procedures in cell cultures or experimental animals. For example, numerous methods of determining ED₅₀ (the dose therapeutically effective in 50 percent of the population) and LD₅₀ (the dose lethal of 50 percent of the population) exist. The dose ratio between therapeutic and toxic effects is the therapeutic index, and it can be expressed as the ratio ED₅₀/LD₅₀. Compositions exhibiting high therapeutic indices are preferred. The data obtained from cell culture assays or animal studies may be used in formulating a range of dosages for human use. The dosage is preferably within a range of concentrations that includes the ED₅₀ with little or no toxicity, and may vary within this range depending on the dosage form employed, sensitivity of the patient, and the route of administration. If administration is not on a daily basis, for example if injections are given every few days or every few months, then more therapeutic agent will be included in each administration, so that daily release of the agent is adequate to meet therapeutic needs.

The pharmaceutical composition may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) routes. Other routes, e.g., intra-articular, may also be used. Such compositions may be prepared by any known method, for example by admixing the active ingredient with the carrier(s) or excipient(s) under sterile conditions.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions). Suitable excipients for tablets or hard gelatine capsules include lactose, maize starch or derivatives thereof, stearic acid or salts thereof. Suitable excipients for use with soft gelatine capsules include for example vegetable oils, waxes, fats, semi-solid, or liquid polyols etc. For the preparation of solutions and syrups, excipients which may be used include for example water, polyols and sugars. For the preparation of suspensions, oils (e.g. vegetable oils) may be used to provide oil-in-water or water-in-oil suspensions. In certain situations, delayed release or enteric-coated preparations may be advantageous, for example to decrease gastric residence time and thereby reduce degradation of the pharmaceutical composition en route to the lower GI tract.

Pharmaceutical compositions adapted for rectal administration may be presented as suppositories or enemas. Pharmaceutical compositions adapted for nasal administration wherein the carrier is a solid include a coarse powder having a particle size for example in the range 20 to 500 microns which is administered in the manner in which snuff is taken, i.e., by rapid inhalation through the nasal passage from a container of the powder held close up to the nose. Suitable compositions wherein the carrier is a liquid, for administration as a nasal spray or as nasal drops, include aqueous or oil solutions of the active ingredient. Pharmaceutical compositions adapted for administration by inhalation include fine particle dusts or mists which may be generated by means of various types of metered dose pressurized aerosols, nebulizers or insufflators.

Pharmaceutical compositions adapted for topical administration may be formulated as ointments, creams, suspensions, lotions, powders, solutions, pastes, gels, sprays, aerosols or oils. When formulated in an ointment, the therapeutic agent may be employed with either a paraffinic or a water-miscible ointment base. Alternatively, the therapeutic agent may be formulated in a cream with an oil-in-water cream base or a water-in-oil base. Pharmaceutical compositions adapted for topical administration to the eye include eye drops wherein the therapeutic agent is dissolved or suspended in a suitable carrier, especially an aqueous solvent. Pharmaceutical compositions adapted for topical administration in the mouth include lozenges, pastilles and mouth washes. Pharmaceutical compositions adapted for transdermal administration may be presented as discrete patches intended to remain in intimate contact with the epidermis of the recipient for a prolonged period of time. Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water, alcohols, polyols, glycerine and vegetable oils, for example. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets. The pharmaceutical compositions may contain preserving agents, solubilizing agents, stabilizing agents, wetting agents, emulsifiers, sweeteners, colorants, odorants, salts (substances of the present invention may themselves be provided in the form of a pharmaceutically acceptable salt), buffers, coating agents or antioxidants.

The compositions may also be included in a kit. The kit can include, in non-limiting aspects, a pharmaceutical composition comprising a therapeutic agent, instructions for administration and/or other components. In preferred embodiments, the kit can include a composition ready for administration. Containers of the kits can include a bottle, dispenser, package, compartment, or other types of containers, into which a component may be placed. The container can include indicia on its surface. The indicia, for example, can be a word, a phrase, an abbreviation, a picture, or a symbol. The containers can dispense a pre-determined amount of the component (e.g. compositions of the present invention). The composition can be dispensed in a spray, an aerosol, or in a liquid form or semi-solid form. The containers can have spray, pump, or squeeze mechanisms. In certain aspects, the kit can include a syringe for administering the compositions of the present invention.

Where there is more than one component in the kit (they may be packaged together), the kit also will generally contain a second, third or other additional containers into which the additional components may be separately placed. The kits of the present invention also can include a container housing the components in close confinement for commercial sale. Such containers may include injection or blow-molded plastic containers into which the desired bottles, dispensers, or packages are retained. A kit can also include instructions for employing the kit components as well the use of any other compositions, compounds, agents, active ingredients, or objects not included in the kit. Instructions may include variations that can be implemented. The instructions can include an explanation of how to apply, use, and maintain the products or compositions, for example.

The administration of the compositions of the present invention may be for a “prophylactic” or “therapeutic” purpose, or alternatively can be used for diagnostic purposes. The compositions of the present invention are said to be administered for a “therapeutic” purpose if the amount administered is physiologically significant to provide a therapy for an actual manifestation of the disease. When provided therapeutically, the compound is preferably provided at (or shortly after) the identification of a symptom of actual disease. The therapeutic administration of the compound serves to attenuate the severity of such disease or to reverse its progress. The compositions of the present invention are said to be administered for a “prophylactic” purpose if the amount administered is physiologically significant to provide a therapy for a potential disease or condition. When provided prophylactically, the compound is preferably provided in advance of any symptom thereof. The prophylactic administration of the compound serves to prevent or attenuate any subsequent advance of the disease.

The dosage schedule and amounts effective for therapeutic and prophylactic uses, i.e., the “dosing regimen”, will depend upon a variety of factors, including the stage of the disease or condition, the severity of the disease or condition, the general state of the patient's health, the patient's physical status, age and the like. In calculating the dosage regimen for a patient, the mode of administration also is taken into consideration. The dosage regimen also takes into consideration pharmacokinetics parameters well known in the art, i.e., the rate of absorption, bioavailability, metabolism, clearance, and the like. The state of the art allows the clinician to determine the dosage regimen for each individual patient, therapeutic agent and disease or condition treated. Single or multiple administrations of the compositions of the present invention can be administered depending on the dosage and frequency as required and tolerated by the patient. The duration of prophylactic and therapeutic treatment will vary depending on the particular disease or condition being treated. Some diseases lend themselves to acute treatment whereas others require long-term therapy.

### C. Other Methods of Treatment

The activity of BIGH3 may also be altered by non-pharmaceutical methods and compositions. For example, gene delivery and gene silencing methods may be used to enhance or inhibit the transcription or translation of BIGH3 and/or its downstream effector molecules. Expression of BIGH3 or other molecules may also be altered by increasing or decreasing the stability of mRNA coding for BIGH3 or the other molecule. Although the following discussion pertains primarily to the inhibition of BIGH3 activity, it can be applied to the enhancement of BIGH3 activity with routine modifications, as is well known to the skilled artisan.

The activity of BIGH3 may be altered by “gene silencing methods” which are generally regarded as methods that prevent or decrease the rate of transcription or translation of a protein within a cell. Such gene silencing methods include, but are not limited to antisense technology, RNA inhibition technology (RNAi) and inactivation or degradation of transcription factors required for BIGH3 transcription, etc.

Generally, RNAi technology is limited to tissue-specific or organ-specific areas of the subject using tissue-specific gene promoters or transcription factors. Promoters are nucleic acids that are generally located in the 5′-region of a gene, proximal to the start codon or nucleic acid which encodes untranslated RNA. The transcription of an adjacent nucleic acid segment is initiated at the promoter region. Any suitable promoter may be used to control the production of RNA from the nucleic acid molecules of the invention. Promoters may be those recognized by any polymerase enzyme; for example, promoters may be promoters for RNA polymerase II or RNA polymerase III. Suitable promoters are known in the art and are within the scope of the present invention. Recombinant DNA methods, such as those that might be used to prepare constructs of a tissue-specific promoter operably linked to a coding region coding for messenger RNA are well known in the art.

One example of a construct designed to produce RNAi is a construct where a DNA segment is inserted into a vector such that RNA corresponding to both strands are produced as two separate transcripts. Another example of a construct designed to produce RNAi is a construct where two copies of a DNA segment are inserted into a vector such that RNA corresponding to both strands are again produced. Yet another example of a construct designed to produce RNAi is a construct where two copies of a DNA segment are inserted into a vector such that RNA corresponding to both strands are produced as a single transcript. Expression of one of these DNA segments results in the production of sense RNA while expression of the other results in the production of an anti-sense RNA. Nucleic acid segments designed to produce RNAi need not correspond to the full-length gene or open reading frame. For example, when the nucleic acid segment corresponds to an open reading frame (ORF), the segment may only correspond to part of the ORF (e.g., about 50 nucleotides or even fewer at the 5′ or 3′ end of the ORF).

Ribozymes may also be used for gene silencing. For example, antisense RNA/ribozymes fusions which comprise (1) antisense RNA corresponding to a target gene and (2) one or more ribozymes which cleave RNA (e.g., hammerhead ribozyme, hairpin ribozyme, delta ribozyme, Tetrahymena L-21 ribozyme, etc.) can be used, as well as vectors which express these fusions, methods for producing these vectors, and methods for using these vectors.

Other methods of inhibiting the activity of BIGH3 include the use of antibodies or functional fragments thereof to the BIGH3 receptor or the BIGH3 protein, which may prevent subsequent downstream signaling that normally follows the binding of BIGH3 to its receptor. As used herein, the term “antibody” includes at least monoclonal antibodies and polyclonal antibodies; and “functional fragments” of an antibody is used to mean a portion of an antibody that can bind, to some extent, at least the antigen of the fully intact antibody. “Functional fragments” thus includes molecules that bind more than one antigen, such as, but not limited to a tetramer of single chain fragment of variable region (scFV). Antibodies or functional fragments thereof can be used as antagonists of activity against BIGH3, MMP2, MMP14, or other BIGH3 effector molecules. Use of functional fragments, such as the Fab, Fab′ or F(ab′)2 fragments are often suitable, especially in a therapeutic context, as these fragments are generally less immunogenic than the whole immunoglobulin.

Antibodies are prepared by well-known methods in the art, such as immunizing suitable mammalian hosts in appropriate immunization protocols using BIGH3 peptides, polypeptides or proteins if they are of sufficient length, or, if desired or required to enhance immunogenicity, they can be conjugated to suitable carriers such as bovine serum albumin (BSA), keyhole limpet hemocyanin (KLH), or the like. Anti-peptide antibodies can be generated using synthetic peptides. Synthetic peptides can be as small as 2-3 amino acids in length, and are suitably at least 3, 5, 10, or 15 or more amino acid residues in length. Such peptides can be determined using programs such as DNAStar. The peptides can be coupled to KLH using standard methods and can be immunized into animals such as rabbits. Polyclonal anti-BIGH3, anti-MMP2, anti-MMP14, or other anti-effector molecule peptide antibodies can then be purified, for example using Actigel beads containing the covalently bound peptide.

While the polyclonal antisera produced in this way may be satisfactory for some applications, for pharmaceutical compositions, use of monoclonal preparations is also suitable. Immortalized cell lines which secrete the desired monoclonal antibodies may be prepared using the standard method of Kohler and Milstein or modifications which effect immortalization of lymphocytes or spleen cells, as is generally known. The immortalized cell lines secreting the desired antibodies are screened by immunoassay in which the antigen is the peptide hapten, polypeptide or protein. When the appropriate immortalized cell culture secreting the desired antibody is identified, the cells can be cultured either in vitro or by production in ascites fluid. The desired monoclonal antibodies can then be recovered from the culture supernatant.

The antibodies or fragments, such as scFV fragments, may also be produced by recombinant means. Regions that bind specifically to the desired regions of BIGH3 or the BIGH3 receptor or its downstream effector molecules can also be produced in the context of chimeras with multiple species origin. Humanized and fully human antibodies, such as those identified by phage display or produced by a Xenomouse, are also contemplated. Antibody reagents so created are contemplated for use diagnostically or as stimulants or inhibitors of the activity of BIGH3 or an effector molecule such as, but not limited to MMP2 and MMP14.

### D. Methods of Screening

Because of the role that BIGH3 and its effector molecules play in tissue repair and fibrosis, it is desirable to identify substances that inhibit or enhance their activity in certain circumstances. Accordingly, several embodiments of the present invention relate to methods of screening and/or identifying compounds useful for treating, preventing or preventing the progression of fibrosis. Specifically, the methods of identifying such inhibitory substances comprise (a) providing a test substance to a cell, wherein the cell possesses BIGH3 activity, (b) measuring the amount of BIGH3 activity in the test cell; and (c) comparing the amount of BIGH3 activity in a control cell to which has not been provided the test substance with the amount of BIGH3 activity in the test cell, wherein a decreased amount BIGH3 activity in the test cell, compared to the amount of BIGH3 activity in the control cell, indicates that the test substance inhibits BIGH3 activity.

Other embodiments of the present invention relate to methods of screening and/or identifying compounds useful for facilitating tissue repair. Specifically, the methods of identifying such enhancing or facilitative substances comprise (a) providing a test substance to a cell, wherein the cell possesses BIGH3 activity, (b) measuring the amount of BIGH3 activity in the test cell; and (c) comparing the amount of BIGH3 activity in a control cell to which has not been provided the test substance with the amount of BIGH3 activity in the test cell, wherein a increased amount BIGH3 activity in the test cell, compared to the amount of BIGH3 activity in the control cell, indicates that the test substance enhances BIGH3 activity. The measuring means may be directly correlative or inversely correlative, so long as the measuring means provides the technician with a means of assessing the levels of BIGH3 activity in test cells that can be compared to levels of BIGH3 activity in control cells.

As used herein, the terms “substance”, “agent” and “compound” may be used interchangeably. The types of substances that may be assayed for their ability to inhibit BIGH3 and/or its effector molecules include, but are not limited to, carbohydrates such as monosaccharides, disaccharides, oligosaccharides and polysaccharides, proteins, peptides and amino acids, including, but not limited to, oligopeptides, polypeptides and mature proteins, nucleic acids, oligonucleotides, polynucleotides, lipids, fatty acids, lipoproteins, proteoglycans, glycoproteins, organic compounds, inorganic compounds, ions, and synthetic and natural polymers.

As used herein, “BIGH3 activity” is assessed by direct or indirect means. For example, BIGH3 activity can be directly assessed by measuring or quantifying levels of BIGH3 protein that binds to a receptor, or is produced by a cell; and BIGH3 activity can be indirectly assessed by measuring or quantifying a detectable effect that BIGH3 protein has on a cell. Detectable effects that BIGH3 has on a cell encompass RNA transcription, protein expression or secretion, such as, but not limited to, collagen expression, or the generation of second messenger or intracellular signals. Thus these embodiments also provide methods of identifying substances useful for inhibiting collagen production or accumulation.

As stated previously, BIGH3 is a chemokine, thus BIGH3 activity also includes activities normally associated with chemokines, such as (1) mediating natural immunity; (2) regulating lymphocyte activation, growth and differentiation; (3) regulating immune-related inflammation; (4) stimulating leukocyte growth and differentiation; and (5) stimulating leukocyte movement. Accordingly, these embodiments provide methods of identifying substances which modulate natural immunity, modulate the activation, growth and/or differentiation of lymphocytes or leukocytes, modulate immune related inflammation, and modulate the stimulation of leukocyte movement. A particular embodiment relates to methods of identifying substances that modulate the activation, growth, differentiation or movement of lymphocytes, in particular T-cells.

BIGH3 activity can be assessed by other means that include, but are not limited to, phosphorylation of second messenger molecules, such as phospholipase C, adenylate cyclase and protein kinase C among others, generation of other second messenger signals such as Ca⁺² release, calmodulin binding, inositol triphosphatase activity, and GTPase activating protein (GAP) activity to name a few. Other indirect measures of BIGH3 activity include activation of transcription factors, such as, but not limited to PU.1 and p53, and levels of mRNA of specific transcripts. Other detectable effects of BIGH3 activity encompass assessing the ability of a substance to bind to the BIGH3 receptor, and can be assayed by traditional procedures such as, but not limited to, competitive binding assays. These screens are not limited to means of measuring BIGH3 activity for the purposes of comparing test substances.

As used herein, the measurement of the activity to be assayed, for example, BIGH3 activity, MMP14 activity, etc., can be a relative or absolute measurement. Of course, the measurement of activity may be equal to zero, indicating the absence of activity. The measurement of activity may be a simple value, without any additional measurements or manipulations. Alternatively, the measurement of activity may be expressed as a difference, percentage or ratio of the measured activity to another value, but not limited to, a normal, baseline or standard measurement. The difference may be negative, indicating a decrease in the amount of measured activity. The measurement of activity may also be expressed as a difference or ratio of the activity to itself, measured at a different point in time. The measurement of activity may be determined directly, or the value of the measured activity may be used in an algorithm, with the algorithm designed to correlate the measurement to the level of activity in the cell.

As used herein the term “cell” is used to indicate one or more cells, and can be used interchangeably with the term “cells”, “cell culture” and “cell line.” In addition, the cells used in the screening methods can be isolated cells in an in vitro cell culture, or the cells may be in situ, as part of an organ or tissue; or the cells may be in vivo as part of an organ or tissue in a live subject, such as, but not limited to a mouse, rat, dog or primate. The cells used in the screening methods may also be manipulated, modified, fixed or even lysed at any time during the screening process, for example, subsequent to application of the test substance, but prior to measuring the activity to be assessed. Provided that assayed activity can be measured (e.g., BIGH3 activity, MMP14 activity, etc.), the cells can thus be prokaryotic or eukaryotic, including but not limited to bacterial cells, insect cells, mammalian cells, and even plant cells. A “test cell” is a cell to which a test substance has been applied; and “control cell” is a cell to which the same test substance has not been applied. The control cell may or may not be a genetically, phenotypically or metabolically normal cell, but the control cell should be the same cell type as the test cell. In addition, the screens are not limited to the source, location or identity of the cells used in the screening methods. Indeed, the cells may be isolated from patients through biopsies, lavage procedures, or other techniques, or may be isolated from whole organ or organ systems such as, but not limited to, the lungs, from a subject.

The cells may also be modified prior to their use in the screening methods described herein. For example, the cells may comprise genetic constructs designed to elucidate differences in the tested activity, e.g., BIGH3 activity, MMP14 activity, etc., within the cell. In one assay format, cell lines that contain reporter gene fusions between the open reading frame and any assayable fusion partner may be prepared. Numerous assayable fusion partners are known and readily available including the firefly luciferase gene and the gene encoding chloramphenicol acetyltransferase. Cell lines containing the reporter gene fusions are then exposed to the test substance under appropriate conditions and time. Differential expression of the reporter gene between samples exposed to test substance and control samples identifies substances that can modulate the expression of a nucleic acid encoding BIGH3, or an upstream activator molecule or a downstream effector molecule.

Additional examples of manipulated cells that may be used for screening methods include, but are not limited to, cells that have been transfected, transformed or infected with genetic constructs comprising BIGH3 and/or one of its effector molecules. For example, recombinant replication-deficient adenovirus comprising the BIGH3 gene may be operably linked to a promoter within the framework of the viral genome. Cultured cells or in vivo cells may then be infected with adenovirus and used to screen substances for their ability to inhibit the activity of BIGH3 or one of its downstream effector molecules.

Additional assay formats may be used to monitor the ability of the substance to modulate the expression of a nucleic acid encoding a protein such as a BIGH3, or an upstream or downstream signaling protein. For instance, mRNA expression may be monitored directly by hybridization to nucleic acids. Cell lines are exposed to the substance to be tested under appropriate conditions, and total RNA or mRNA can be isolated by standard procedures such those disclosed in Sambrook et al. (1989). Probes to detect differences in RNA expression levels between cells exposed to the agent and control cells may be prepared from the nucleic acids of the invention. Probes may be designed from the nucleic acids of the invention through methods known in the art. For instance, the G+C content of the probe and the probe length can affect probe binding to its target sequence. Methods to optimize probe specificity are commonly available in Sambrook et al. (1989) or Ausubel et al. (Current Protocols in Molecular Biology, Greene Publishing Co., New York, 1995). Probes may be designed to hybridize selectively with target nucleic acids under conditions that maximize the difference in stability between the probe:target hybrid and potential probe:non target hybrids, such as high stringency conditions, methods of which are well known in the art.

Hybridization conditions are modified using known methods, such as those described by Sambrook et al. and Ausubel et al. as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a porous glass wafer. The glass or silica wafer can then be exposed to total cellular RNA or polyA RNA from a sample under conditions in which the affixed sequences will specifically hybridize. Such glass wafers and hybridization methods are widely available, for example, those disclosed by Beattie (WO 95/11755). By examining for the ability of a given probe to specifically hybridize to an RNA sample from an untreated cell population and from a cell population exposed to the agent, agents can be assayed for their ability to up or down regulate the expression of a nucleic acid encoding the BIGH3, or an upstream or downstream signaling protein.

Application of the teachings of the present invention to a specific problem or environment is within the capabilities of one having ordinary skill in the art in light of the teachings contained herein. Examples of the products and processes of the present invention appear in the following examples.

### EXAMPLE 1

### Macrophage and Fibroblast Cell Culture

Macrophages were derived from human peripheral blood monocytes (monocyte-derived macrophages [MDM]), bronchoalveolar lavage fluids (alveolar macrophages [AM]), or a human monocytic cell line THP-1 (THP-1-derived macrophages [TDM]). To produce MDM, peripheral blood mononuclear cells (PBMC) were isolated from freshly drawn peripheral blood by density gradient centrifugation using Ficoll-Paque (Amersham Biosciences, Piscataway, N.J.) and resuspended in RPMI 1640 medium supplemented with 20% human serum, 10 mM HEPES, pH 7.4, 2 mM L-glutamine, 1 mM sodium pyruvate, 0.1 mM non-essential amino acid mix, 5×10⁻⁵ M 2-ME, and 5 μg/ml gentamicin sulfate. The cells were cultured overnight in 6-well plates (Becton Dickinson, Franklin Lakes, N.J.), in a 5% CO₂ humidified air atmosphere at 37° C. The non-adherent cells were removed and the adherent cells were cultured for an additional five days, and were termed monocyte-derived macrophages. Alveolar macrophages were obtained from broncho-alveolar lavage fluids derived from two adult healthy individuals or from eight patients with interstitial lung disease associated with systemic sclerosis, by the methods reported in Atamas and Luzina. See S. P. Atamas et al. (1999) Arthritis Rheum. 42:1168-1178; I. G. Luzina et al. (2002) Am J Respir Cell Mol Biol. 26:549-557; I. G. Luzina et al. (2003) Arthritis Rheum. 48:2262-2274. The protocols for drawing blood and for broncho-alveolar lavage procedures were approved by the University of Maryland Institutional Review Board.

Human monocytic line THP-1 was obtained from the American Type Culture Collection (ATCC, Manassas, Va.) and maintained in the same medium, except that 10% FBS was used instead of human serum. The THP-1-derived macrophages were obtained by stimulating these cells with 200 nM/ml phorbol-12-myristate-13-acetate (PMA) purchased from Cell Signaling (Danvers, Mass.). Four primary pulmonary fibroblast cultures (PF1-PF4) derived from different adult healthy donors were purchased from Cambrex (Walkersville, Md.) and each tested separately in independent experiments. Fibroblast cultures were maintained in T75 culture flasks as previously described. S. P. Atamas et al. (2003) Am J Respir Cell Mol Biol. 29:743-749; I. G. Luzina et al. (2006) J Cell Physiol. 206:221-228; S. P. Atamas et al. (2002) J Immunol. 168:1139-1145. In all experiments fibroblast cell lines were tested in passages three to seven.

### EXAMPLE 2

### Apoptotic and Necrotic Cellular Debris

Jurkat cells (human T cell line) were purchased from ATCC and maintained according to the supplier's recommendations. Apoptosis of Jurkat cells was induced by incubation with 0.5 μg/ml staurosporine (Sigma Aldrich, St. Louis, Mo.) at 37° C. for 6-8 h or, alternatively, by exposure to UV irradiation at 90 mJ/cm² followed by culture for 3-4 h. The percentage of apoptotic cells was quantified by flow-cytometry analysis by using Annexin V and propidium iodide staining (Sigma Aldrich, St Louis, Mo.) and was within 70-80%. Necrotic debris was generated by three cycles of freezing-thawing involving freezing the cells in liquid nitrogen and then thawing them at 37° C.

### EXAMPLE 3

### Phagocytosis Assays

Jurkat cells were labeled with the dye TAMRA (5-[and 6-]carboxy tetramethyl rhodamine succinimidyl ester) (Molecular Probes, Eugene, Oreg.). TAMRA-labeled cells were added to cultured macrophages at a ratio of 5:1 and incubated for 2 h at 37° C. At the end of the incubation period, the monolayer was vigorously washed with ice-cold phosphate-buffered saline to remove unbound and bound but un-engulfed apoptotic cells. The phagocytosis was assessed by fluorescent microscopy. The percentage of macrophages that ingested TAMRA-labeled apoptotic cells was determined as the percent phagocytosis (number of macrophages, per 100, that ingested at least one apoptotic cell) in three different wells. Also, the conditioned supernatant media from these cell cultures were collected and used for stimulation of fibroblast cultures as described below.

### EXAMPLE 4

### Western Blotting

Preparation of cell lysates, immunoprecipitation of BIGH3 protein, normalization of protein concentration in the samples with BioRad assays, electrophoretic separation and Western blotting were performed as previously described by Atamas. S. P. Atamas et al. (2003) Am J Respir Cell Mol Biol. 29:743-749. Goat antibodies for BIGH3 were purchased from R&D Systems (Minneapolis, Minn.). Goat antibodies for MMP14 were purchased from Santa-Cruz Biotechnology (Santa Cruz, Calif.). Western blotting assays for collagen were performed using rabbit affinity purified anti-collagen type I antibody (Rockland, Gilbertsville, Pa.).

### EXAMPLE 5

### Collagen Production Assays

Production of collagen was measured in cell cultures utilizing the metabolic labeling of collagen with ¹⁴C-proline as described by Luzina. I. G. Luzina et al. (2006) Am J Respir Cell Mol Biol. 35:298-305; I. G. Luzina et al. (2006) Arthritis Rheum. 54: 2643-2655. Briefly, fibroblast monolayers were pulsed with L-[U-¹⁴C]-proline (Amersham Biosciences, Piscataway, N.J.) at 1 μCi/ml for the final 12 hours of incubation. Purified bacterial collagenase type III was purchased from Sigma Aldrich (St. Louis, Mo.). Fibroblasts were then ruptured by repeated freeze-thawing, and part of each sample digested with collagenase type III. The samples were pelleted with 20% TCA containing 0.1% L-proline, and then the pellets were resuspended and washed twice with 5% TCA and 95% ice-cold ethanol. The samples were assayed in a liquid scintillation counter in order to determine the amount of collagenase-digestible and non-digestible ¹⁴C-labeled protein. Alternatively, collagen protein levels in cell culture supernatants were measured in Western blotting assays as described above. The activity of the COL1A2 promoter was measured in primary fibroblasts transfected with collagen promoter-chloramphenicol acetyltransferase (CAT) reporter constructs as described by Luzina. I. G. Luzina et al. (2006) Am J Respir Cell Mol Biol. 35:298-305.

### EXAMPLE 6

### ELISA

ELISA kit for TGF-β1 was purchased from R&D Systems (Minneapolis, Minn.) and assays performed following the manufacturer's recommendations. Fibroblast culture supernatants and whole cell lysates were activated by acidification prior to the assay to quantify total (active and latent) TGF-β1. All samples were assessed in duplicates. Low-serum cell culture medium containing 0.5% dialyzed FBS had no detectable TGF-β1 and was used as a negative control in these assays.

### EXAMPLE 7

### Real-Time PCR Quantification of mRNA Levels

Total RNA purification, reverse transcription, and real-time PCR were performed using LightCycler (Roche, Indianapolis, Ind.), as previously described by Atamas. S. P. Atamas et al. (2003) Am J Respir Cell Mol Biol. 29:743-749. Quantification of internal control 18S ribosomal RNA was performed as reported previously by Luzina. I. G. Luzina et al. (2006) J Cell Physiol. 206:221-228. The PCR reaction mixture included the recommended components of the FastStart DNA Master Hybridization Probes Hot Start Reaction Mix (Roche, Indianapolis, Ind.). The fold difference in gene expression relative to 18S ribosomal RNA between treated and untreated cultures was calculated using the 2^{-ΔΔC}t method. I. G. Luzina et al. (2006) Arthritis Rheum. 54: 2643-2655. The primers and the hybridization probes for COL1A2 mRNA were designed and prepared by TIB Molbiol (Adelphia, N.J.). The primers for COL1A2 mRNA were: forward, 5′-GAT GGT GAA GAT GGT CCC ACA GG-3′ (SEQ ID NO: 1) and reverse, 5′-GGT CGT CCG GGT TTT CCA GGG T-3′ (SEQ ID NO: 2). The hybridization probes were labeled with fluorescein at the 3′-terminus (3FL) of one probe and with LightCycler Red at the 5′-terminus (5LC) of the other probe. The probes were 3FL 5′-TTC CAA GGA CCT GCT GGT GAG CCT-3′ (SEQ ID NO: 3) and 5LC 5′-TGA ACC TGG TCA AAC TGG TCC TGC AG-3′ (SEQ ID NO: 4). BIGH3-specific primers and PU.1-specific primers were designed and tested for specificity by SuperArray (Frederick, Md.), and their specificity has been additionally confirmed in preliminary experiments. Real-time PCR assays (RT2 Profiler™, SuperArray, Frederick, Md.) were utilized to measure expression of 84 genes related to extracellular matrix in fibroblasts, following the manufacturer's recommendations.

### EXAMPLE 8

### Nucleofection of Primary Fibroblast Cultures

Nucleofection with collagen promoter-chloramphenicol acetyltransferase (CAT) reporter constructs (I. G. Luzina et al. (2006) Am J Respir Cell Mol Biol. 35:298-305), MMP14- or p53-encoding constructs (under control of CMV promoter, OriGene Technologies, Rockville, Md.), siRNA directed against PU.1 or non-targeting control siRNA (both from Santa-Cruz Biotechnology, Santa Cruz, Calif.) was performed using Basic Nucleofector kit reagents from Amaxa (Gaithersburg, Md.), following manufacturer's recommendations. Transfected fibroblasts were cultured for 48 h before treatment with 300 ng/ml BIGH3. The efficiency of PU.1 depletion was assessed by measuring the levels of PU.1 mRNA by Q-PCR.

### EXAMPLE 9

### Profiling of Gene Expression with DNA Arrays

Expression of 367 genes for cytokines and cytokine receptors in macrophages was profiled with cDNA macroarrays (SuperArray, Frederick, Md.) at 0, 2, 6, 12, 24 h of exposure to apoptotic debris. Developed membranes were scanned and hybridization intensities for each spot were measured using Image Quant software (Molecular Dynamics, Sunnyvale, Calif.) and background subtracted. Numeric spot density data were exported into a spreadsheet software for data analyses. Results were confirmed by real-time PCR assays for selected genes.

### EXAMPLE 10

### Macrophage-Fibroblast Co-Cultures and Conditioned Medium Experiments

Fibroblasts were seeded in 6 well-tissue culture plates (Becton Dickinson, Franklin Lakes, N.J.) at a sub-confluent density of 150,000 cells/well and grown for 24 h in the same conditions as described above, except that low-serum 1640 RPMI medium supplemented with 50 μM ascorbic acid, and 50 μM BAPN (β-aminopropionitrile) was used. Then, macrophages were added to each well at a concentration of 250,000 cells/well for additional 24 h, followed by adding 1.5 mln apoptotic Jurkat cells for 2 h. After 2 h, the adherent cells were washed to remove non-ingested apoptotic cells and fresh medium was added for additional 24 h before analyzing these cultures for collagen or cytokine production. In separate experiments, fibroblasts were stimulated with the conditioned media collected from macrophage cultures following phagocytosis assays as described above. Fibroblast proliferation was tested as described in S. P. Atamas et al. (2002) J Immunol. 168:1139-1145. Briefly, after 5 to 7 days of co-culture, macrophages were removed and fibroblast proliferation tested using CellTiter AQueous 96 Non-Radioactive Cell Proliferation Assay (Promega, Madison, Wis.) per manufacturer's recommendations.

Results indicated that collagen production but not proliferation of fibroblasts is upregulated in co-cultures with macrophages following ingestion of apoptotic cells. TAMRA-labeled apoptotic cells were co-cultured with either THP-1 derived macrophages (TDM), monocyte-derived macrophages (MDM), or alveolar macrophages (AM). On average, 31±6% of macrophages had engulfed at least one apoptotic Jurkat T cell. There was no significant difference in the percent phagocytosis between the three types of macrophages (p>0.05, one-way ANOVA). Because fibroblast proliferation and collagen turnover jointly define fibrosis, we tested whether ingestion of apoptotic debris affects these two processes in the macrophage-fibroblast co-cultures. Phagocytosis of TDM, MDM, or AM did not affect proliferation rates in the macrophages-fibroblast co-cultures (p>0.05, two-tailed Student's t-test comparing co-cultures of primary pulmonary fibroblasts with macrophages that engulfed or did not engulf apoptotic cells).

The initial experiments were performed with TDM. The results suggested that these non-activated macrophages or those exposed to necrotic Jurkat cells failed to affect collagen production in co-cultures with pulmonary fibroblasts (FIG. 1). However, ingestion of the apoptotic cells by TDM caused a significant increase in collagen production in co-cultures, as judged by ¹⁴C-proline incorporation and Western blotting assays (FIG. 1). These experiments were repeated on thirteen independent occasions, in duplicates or triplicates, using ¹⁴C-proline incorporation, and on eight independent occasions using Western blotting for collagen in primary fibroblast cultures from four different unrelated donors, with consistent results. These observations suggested that ingestion of apoptotic debris by macrophages may have a profibrotic effect. Further experiments included monocytes-derived and alveolar macrophages.

Co-culturing primary fibroblasts with MDM did not significantly influence collagen production in fibroblasts when compared with fibroblasts cultured alone (p>0.05, FIG. 2). Co-culturing MDM that have ingested apoptotic cells with fibroblasts caused a significant increase in collagen production (FIG. 2). These results were consistently observed in two independent experiments using primary fibroblast cultures from four different unrelated donors. A different pattern of modulation of collagen production was observed when primary fibroblasts were co-cultured with AM from two healthy volunteers or eight patients with interstitial lung disease. Collagen production was increased when primary fibroblasts were co-cultured with non-stimulated AM (FIG. 2). There was no further increase in collagen production in fibroblast co-cultures with AM that have ingested apoptotic cells (FIG. 2). No statistically significant difference was observed between AM macrophages from healthy donors and from patients with interstitial lung disease in these relatively small subsets of volunteers (not shown, p>0.05). These observations suggested that macrophages derived from monocytes (TDM or MDM) respond to apoptotic cells by increasing their profibrotic potential, whereas alveolar macrophages appear to be pre-activated by the pulmonary milieu in their ability to induce accumulation of collagen, and fail to further upregulate collagen levels following exposure to apoptotic cells.

### EXAMPLE 11

### Transwell Assays

To determine whether direct cell-to-cell contacts are necessary to mediate the effects of macrophages on fibroblasts, or whether soluble profibrotic factors are sufficient for the interactions between these two cell types, Transwell assays were performed. In these assays, macrophages were co-cultured with fibroblasts with or without separation with a semipermeable membrane (FIG. 3A), or, separately, the conditioned supernatant from the macrophage cultures were transferred into fibroblast cultures and the effect on collagen production was evaluated (FIG. 3B). In the co-cultures, macrophages were separated from fibroblast monolayers by a semipermeable membrane with 3.0 μm pore size in the 6 well-Transwell plates (Corning Costar Corp., Cambridge, Mass.). Primary lung fibroblasts were seeded in the lower chamber whereas macrophages that engulfed apoptotic cells were placed in the upper chamber, using the same cell culture medium as described above. These co-cultures were incubated for 24 hours before analyzing the levels of collagen or cytokines.

Results indicated that the profibrotic effect of macrophages on fibroblasts is mediated by soluble factors. Macrophages that have ingested apoptotic debris stimulated collagen production in fibroblast monolayers even when separated by a Transwell membrane (FIG. 3A). Also, the conditioned medium from macrophages that have ingested apoptotic debris stimulated collagen production in fibroblast monolayers (FIG. 3B). These observations suggested that soluble factors produced by macrophages following ingestion of apoptotic debris drive the increase in collagen production. To further investigate this mechanism, we considered a well-known upregulation in production of TGF-β, a potent profibrotic cytokine, by macrophages following ingestion of apoptotic debris. V. A. Fadok et al. (1998) J Clin Invest. 101:890-898. ELISA assays revealed that indeed the levels of total TGF-β1 were increased in macrophage cultures 2 h and 24 h of exposure to apoptotic debris (FIG. 4). Of important notice, the levels of TGF-β1 production are consistent with the previous observation by Fadok et al., and are not sufficient to directly drive collagen production in fibroblasts, particularly because no active TGF-β was detected in the co-cultures. See V. M. Kahari et al. (1990) J Clin Invest. 86:1489-1495; A. Fine & R. H. Goldstein (1987) J Biol Chem. 262:3897-3902; I. G. Luzina et al. (2006) Am J Respir Cell Mol Biol. 35:298-305. Also, TGF-β is known to increase collagen production transcriptionally, but our real-time PCR experiments for COL1A2 mRNA revealed no increase in collagen mRNA in the macrophage-fibroblast co-cultures. Therefore, results indicated that factors other than TGF-β contribute to upregulation of collagen production by the macrophages following ingestion of apoptotic debris.

### EXAMPLE 12

### BIGH3 is Generated by Macrophages Following Exposure to Apoptotic Cells

Transcriptomic profiling of macrophages using cDNA arrays revealed that out of nearly 400 cytokine, chemokine, and their receptor genes represented on the array, only one, transforming growth factor β-induced (TGFBI), also known as BIGH3, was consistently increased shortly (2 h) following ingestion of apoptotic debris. Reverse transcriptase-real-time PCR assays confirmed that the steady-state levels of BIGH3 mRNA increased significantly when THP-1 derived (TDM), and monocyte-derived macrophages (MDM) engulfed apoptotic cells compared to non-stimulated macrophages (FIG. 5A). MDM were tested on two independent occasions, and TDM and AM were tested on four independent occasions, in duplicate cultures, with consistent results. Further experiments defined the dynamics of increase in levels of BIGH3 mRNA in macrophage cultures following ingestion of apoptotic debris (FIG. 5B). The latter experiments were repeated on two different occasions with consistent results (p<0.5, one-way ANOVA). In the THP-1 derived macrophages, engulfment of apoptotic Jurkat T cells stimulated an increase in BIGH3 protein levels, as judged by the density of the collagen bands in Western blotting analyses (FIG. 5C). No differences in steady-state levels of BIGH3 mRNA and BIGH3 protein were observed between activated and control cultures of alveolar macrophages (AM) (FIGS. 5A, D). The Western blotting experiments were repeated on two different occasions in each of these macrophage types.

Others have previously reported that so-called alternatively activated macrophages express elevated levels of BIGH3. Gratchev et al. (2001) Scand J Immunol. 53:386-392. Another group also reported that such macrophages stimulate collagen production in co-cultures with fibroblasts. E. Song et al. (2000) Cell Immunol. 204:19-28. Therefore, a possibility was considered that phagocytosis of apoptotic debris may convert macrophages toward an alternative phenotype that is characterized by the cell surface expression of CD163 and CD206. V. Porcheray et al. (2005) Clin Exp Immunol. 142:481-489. We conducted flow-cytometric analyses of TDM and MDM macrophages before and after phagocytosis of apoptotic debris and observed no increase in the cell surface expression of CD163 and CD206 at 24 h post incubation, as judged by lack of change in the mean fluorescence intensity. Therefore, ingestion of apoptotic cells causes a selective increase in the expression levels of BIGH3 without complete alternative activation of macrophages.

### EXAMPLE 13

### BIGH3 Upregulates Collagen Levels in Primary Fibroblasts

The finding of increased BIGH3 production by macrophages following ingestion of the apoptotic debris suggested that this cytokine may directly regulate collagen accumulation in fibroblast cultures. To test this possibility, fibroblast cultures were incubated for various times with various doses of recombinant human BIGH3 (rhBIGH3), and the levels of collagen accumulation were measured. Results indicated that BIGH3 directly regulated collagen production in cultured primary pulmonary fibroblasts in a dose- (FIGS. 6A, B) and time- (FIGS. 6C, D) dependent fashion. These experiments were repeated on at least two occasions, in duplicates, for each of the four primary fibroblast cultures, with consistent results.

Further experiments were performed to determine whether the increase in collagen levels stimulated by BIGH3 was due to transcriptional upregulation of the collagen gene expression. To test whether collagen gene expression was transcriptionally upregulated by BIGH3, COL1A2 mRNA steady state levels were measured by Q-PCR and showed no differences in steady-state levels of collagen α2(I) mRNA between BIGH3-treated and control cultures. The response of the COL1A2 promoter-CAT reporter constructs was evaluated and no differences were observed. Finally, activation of DNA binding by transcription factors known to regulate the activity of collagen gene promoter (Smad3/4, Sp1, AP1, Ets) was tested using transcription factor array approach (TranSignal™ Protein/DNA system, Panomics, Fremont, Calif.), and again, no differences were detected. These results showed that the observed upregulation of collagen protein levels by BIGH3 (see FIG. 6) was not due to transcriptional regulation at the level of the collagen gene promoter.

### EXAMPLE 14

### BIGH3 Inhibits Expression of MMP14

Experiments were performed to determine whether BIGH3 upregulates collagen levels in fibroblast cultures by attenuating collagen turnover. To address such a possibility, experiments were performed in which primary fibroblasts were incubated for 6 h with or without 300 ng/ml BIGH3. The expression levels of 84 genes related to connective tissue biology were analyzed using reverse transcriptase—real time PCR approach (RT² Profiler™ SuperArray, Frederick, Md.). Expression of matrix metalloproteinase (MMP) 14 was consistently decreased following stimulation with BIGH3 (FIG. 7A). Western blotting analyses confirmed that MMP14 protein levels decreased significantly in fibroblast cultures activated with BIGH3 for 24 h or 48 h, compared to non-activated fibroblasts (FIG. 7B). These experiments were repeated on three independent occasions with primary fibroblast cultures from different donors, with consistent results. MMP14 is a critical factor for collagen turnover by fibroblasts. H. Lee et al. (2006) Mol Biol Cell. 17:4812-4826. This decrease in MMP14 levels (FIG. 7) is consistent with the increase in collagen levels (FIG. 6), as collagen turnover is likely to be downregulated due to lower levels of MMP14. To further confirm the inverse link between the levels of MMP14 and collagen, primary fibroblasts were transfected with either an MMP14-encoding plasmid construct or the corresponding “blank” plasmid (FIG. 8A). As expected, the levels of MMP14 following the transfection were increased and the levels of collagen reciprocally decreased (FIG. 8A).

### EXAMPLE 15

### Analyses of DNA Binding by Transcription Factors

Nuclear extracts from BIGH3-activated and control fibroblast cultures were prepared using nuclear extraction kit from Active Motif (Carlsbad, Calif.) and adjusted for total protein content using Bio-Rad assays. DNA binding by 345 different transcription factors was evaluated using protein/DNA TranSignal™ system (Panomics, Redwood City, Calif.), following the manufacturer's recommendations.

Results indicated that regulation of MMP14 and collagen by BIGH3 is dependent on PU1 and p53. Parallel semi-quantitative screening of DNA binding by transcription factors revealed significant upregulation of DNA binding by PU.1 and downregulation of DNA binding by p53 in response to stimulation of fibroblasts with 300 ng/ml rhBIGH3. These observations suggested that PU.1 is a repressor, whereas p53 is an activator of MMP14 expression. To test whether PU.1 is involved in the regulation of MMP14 and collagen levels, PU.1 expression in fibroblasts was inhibited using siRNA transfection technique. Real-time PCR analyses revealed that levels of PU.1 mRNA were decreased three fold in PU.1 siRNA-transfected fibroblasts compared to control siRNA-transfected fibroblasts. Simultaneously, expression levels of MMP14 increased and levels of collagen decreased in PU.1 siRNA-transfected fibroblasts (FIG. 8B). Transient transfection of fibroblast cultures with p53-encoding plasmid construct was performed to determine whether overexpression of p53 leads to increase in MMP14 and a decrease in collagen protein levels. As expected, MMP14 protein levels increased and collagen levels decreased in fibroblasts transfected with p53-encoding plasmid (FIG. 8C). These observations suggest that PU.1 and p53 are involved in the regulation of MMP14 and collagen levels in fibroblasts.

All publications and patents mentioned in the above specification are herein incorporated by reference. The above description, drawings and examples are only illustrative of preferred embodiments which achieve the objects, features and advantages of the present invention. It is not intended that the present invention be limited to the illustrative embodiments. Any modification of the present invention which comes within the spirit and scope of the following claims should be considered part of the present invention.

What is claimed is:

1. A method of treating fibrosis in a subject in need of treatment thereof, said method comprising inhibiting the activity of transforming growth factor beta induced (BIGH3).

2. The method of claim 1, wherein said inhibiting the activity of BIGH3 comprises inhibiting the binding of BIGH3 to its receptor.

3. The method of claim 1, wherein said inhibiting the activity of BIGH3 comprises inhibiting the expression of BIGH3.

4. The method of claim 1, wherein said inhibiting the activity of BIGH3 comprises inhibiting the activity of an effector molecule of BIGH3.

5. The method of claim 4, wherein said effector molecule is selected from the group consisting of PU.1 transcription factor and p53 transcription factor.

6. The method of claim 1, wherein said inhibiting the activity of BIGH3 comprises RNA antisense inhibition.

7. The method of claim 1, wherein said inhibiting the activity of BIGH3 comprises administering a pharmaceutically effective amount of a BIGH3 antagonist to the subject.

8. A method of treating a disorder that results in fibrosis or sclerosis, in a subject in need of such treatment, comprising administering a composition comprising an inhibitor of BIGH3 activity, wherein said composition is administered in an amount effective to reduce the rate of fibrosis or sclerosis.

9. The method of claim 8 wherein said disorder is selected from skeletal muscle fibrosis, irradiation-induced fibrosis, autoimmune-related fibrosis, cardiovascular fibrosis, arteriosclerotic disorders, pulmonary fibrosis, adult respiratory distress syndrome, inflammatory disorders, scleroderma, cirrhosis, keloids, adhesions and hypertrophic scars.

10. The method of claim 8, wherein said composition is administered topically.

11. The method of claim 8, wherein said composition is administered parenterally.

12. The method of claim 8, wherein said disorder is a dermal fibrosis and said composition is administered topically.

13. The method of claim 8, wherein said disorder is pulmonary fibrosis and said composition is administered by inhalation.

14. The method of claim 8, wherein said disorder is rheumatoid arthritis and said composition is administered by intra-articular injection.

15. The method of claim 8, wherein said composition is administered directly to the affected anatomic site.

16. The method of claim 8, wherein said composition is administered prophylactically.

17. A method of preventing fibrosis or sclerosis in a subject in need of such treatment, comprising administering a composition comprising an inhibitor of BIGH3 activity, wherein said composition is administered in an amount effective to reduce the formation of fibrotic or sclerotic tissue that would occur in the absence of such treatment.

18. The method of claim 17, wherein said fibrosis or sclerosis is due to keloid formation, hypertrophic scar formation, or adhesion formation.

19. The method of claim 17, wherein said fibrosis or sclerosis is selected from skeletal muscle fibrosis, irradiation-induced fibrosis, autoimmune-related fibrosis, cardiovascular fibrosis, arteriosclerotic disorders, pulmonary fibrosis, adult respiratory distress syndrome, inflammatory disorders, scleroderma, or cirrhosis.

20. The method of claim 17, wherein said composition is administered prior to scheduled surgery.
